(19) United States
(12) Patent Application Publication
(10) Pub. No.: US 2004/0138139 A1
(43) Pub. Date: Jul. 15, 2004

(54) Novel compounds

(76) Inventors: Patrick Camilleri, Harlow (GB); Anthony John Kirby, Cambridge (GB); Christele Perrin, Cambridge (GB); Gael Alain Bertrand Ronsin, Cambridge (GB); Philippe Guedat, Lyon (FR)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/451,030

(22) PCT Filed: Dec. 17, 2001
(86) PCT No.: PCT/EP01/14821

(30) Foreign Application Priority Data
Dec. 19, 2000 (GB) 0031068.0

(51) Int. Cl. A61K 38/06; A61K 38/05; A61K 38/04; C07K 5/04

(52) U.S. Cl. 514/17; 514/18; 514/19; 530/330; 530/331; 548/339.1; 548/495; 564/152

(57) ABSTRACT

Diaminodicarboxylic acid:peptide gemini surfactant compounds are disclosed. Uses of the diaminodicarboxylic acid: peptide-based gemini surfactant compounds and methods for their production are also disclosed.

This invention relates to newly identified diaminodicarboxylic acid:peptide-based gemini surfactant compounds, to the use of such compounds and to their production. The invention also relates to the use of diaminodicarboxylic acid:peptide-based gemini compounds to facilitate the transfer of compounds into cells for drug delivery.

Surfactants are substances that markedly affect the surface properties of a liquid, even at low concentrations. For example surfactants will significantly reduce surface tension when dissolved in water or aqueous solutions and will reduce interfacial tension between two liquids or a liquid and a solid. This property of surfactant molecules has been widely exploited in industry, particularly in the detergent and oil industries. In the 1970s a new class of surfactant molecule was reported, characterised by two hydrophobic chains with polar heads which are linked by a hydrophobic bridge (Deinega,Y et al., Kolloidn. Zhz. 36, 649, 1974). These molecules, which have been termed “geniini” (Menger, F M and Littau,C A, J.Am.Chem.Soc. 113, 1451, 1991), have very desirable properties over their monomeric equivalents. For example they are highly effective in reducing interfacial tension between oil and water based liquids and have a very low critical micelle concentration.

Cationic surfactants have been used inter alia for the transfection of polynucleotides into cells in culture, and there are examples of such agents available commercially to scientists involved in genetic technologies (for example the reagent Tfx™-50 for the transfection of eukaryotic cells available from Promega Corp. WI, USA).

The efficient delivery of DNA to cells in vivo, either for gene therapy or for antisense therapy, has been a major goal for some years. Much attention has concentrated on the use of viruses as delivery vehicles, for example adenoviruses for epithelial cells in the respiratory tract with a view to corrective gene therapy for cystic fibrosis (CF). However, despite some evidence of successful gene transfer in CF patients, the adenovirus route remains problematic due to inflammatory side-effects and limited transient expression of the transferred gene. Several alternative methods for in vivo gene delivery have been investigated, including studies using cationic surfactants. Gao,X et al. (1995) Gene Ther. 2, 710-722 demonstrated the feasibility of this approach with a normal human gene for CF transmembrane conductance regulator (CFTR) into the respiratory epithelium of CF mice using amine carrying cationic lipids. This group followed up with a liposomal CF gene therapy trial which, although only partially successful, demonstrated the potential for this approach in humans (Caplen, N J. et al., Nature Medicine, 1, 3946, 1995). More recently other groups have investigated the potential of other cationic lipids for gene delivery, for example cholesterol derivatives (Oudrhiri,N et al. Proc.Natl.Acad.Sci. 94, 1651-1656, 1997). This limited study demonstrated the ability of these cholesterol based compounds to facilitate the transfer of genes into epithelial cells both in vitro and in vivo, thereby lending support to the validity of this general approach.

These studies, and others, show that in this new field of research there is a continuing need to develop novel low-toxicity surfactant molecules to facilitate the effective transfer of polynucleotides into cells both in vitro for transfection in cell-based experimentation and in vivo for gene therapy and antisense treatments. The present invention seeks to overcome the difficulties exhibited by existing compounds.

Recently a number of peptide-based gemini surfactants having gene transfection properties were disclosed in WO99/29712 (SmithKline Beecham).

The invention relates to diaminodicarboxylic acid:peptide-based gemini compounds having a diaminodicarboxylic acid backbone and conforming to the general structure of formula (I):

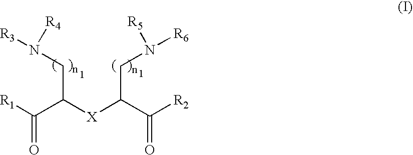

where X=(CH₂)ₙ₂, n2 is 1 to 8 and n1 is 0; or

where X=NHC(O)(CH₂)ₙ₃C(O)NH, n3 is 1 to 8 and n1 is 2 to 4; or

where X=(CH₂)ₙ₄NHC(O)(CH₂)ₙ₅C(O)NH(CH₂)ₙ₄, n4 is 2 to 4, n5 is 1 to 8 and n1 is 0; and

where R₃, R₄, R₅ and R₆ is hydrogen; or

where R₃ and R₅ is hydrogen and R₄ and R₆ which may be the same or different are peptide groups formed from one or more amino acids linked together by amide (CONH) bonds and further linked to the diaminodicarboxylic acid backbone by amide bonds, in a linear or branched manner, having the general formula (II):

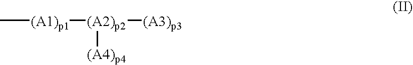

where the values for p1 and p2, which may be the same or different, are from 0 to 5, preferably 1; and

the values for p3 and p4, which may be the same or different, are from 0 to 5, preferably 0;

A1, A3 and A4, which may be the same or different, is an amino acid selected from serine, lysine, omithine, threonine, histidine, cysteine, arginine and tyrosine; and

A2 is an amino acid selected from lysine, omithine and histidine; or

where R₄ and R₆, which may be the same or different, is a group having the formula (III):

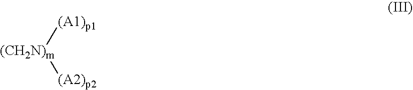

and R₃ and R₅, which may be the same or different, is a group having the formula (IV):

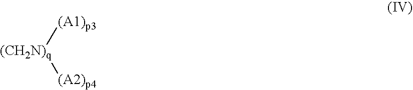

where p1, p2, p3, and p4 which may be the same or different, are 0 to 5;

and where m is 0 to 5; q is 1 to 5;

and where A1 to A4 are as defined above;

and R₁ and R₂ are saturated or unsaturated aminohydrocarbyl groups having up to 32 carbon atoms and linked to the diaminodicarboxylic acid backbone by an amide bond; or

a salt, preferably a pharmaceutically acceptable salt thereof.

Preferably, the compound is symmmetrical, that is R₁ and R₂ are the same, R₃ and R₅ are the same, and R₄ and R₆ are the same.

In a preferred embodiment A1 is serine or threonine, preferably serine. Preferably A3 and A4 are lysine, orithine, histidine or arginine.

In a further preferred embodiment the aminohydrocarbyl group is selected from:

—NH(CH₂)₁₁CH₃

—NH(CH₂)₁₃CH₃

—NH(CH₁₎₁₅CH₃

—NH(CH₂)₁₇CH₃

—NH(CH₂)₁₉CH₃

—NH(CH₂)₂₃CH₃

—NH(CH₂)₈CH═CH(CH₂)₅CH₃

—N(CH₂)₈CH═CH(CH₂)₇CH₃

—NH(CH₂)₈CH═CHCH₂CH═CH(CH₂)₄CH₃

—NH(CH₂)₈ (CH═CH(CH₂)₃ CH₃

—NHC₄CH═CH(C₂CH═CM3(CH₂)₄CH₃

—NH(CH₂)₈CH═CH(CH₂)₅CH₃ Trans

—NH(CH₄)₈CH═CH(CH₂)₇CH₃ Trans

—NH(CH₂)₉CHCH₃(CH₂)₇CH₃

—NHC₂CHOH(C H₂)₂CH₃

—N((CH₂)₁₅CH₃)₂

—NH(CH₂)₈C≡C(CH₂)₇CH₃

—NH(CH₂)₁₁CH₃

—NH(CH₂)₁₃CH₃

—NH(CH₂)₁₅CH₃

—NH(CH₂)₁₇CH₃

—NH(CH₂)₁₉CH₃

—NH(CH₂)₂₃CH₃

—NH(CH₂)₈CH═CH(CH₂)₅CH₃

—NH(CH₂)₈CH═CH(CH₂)₇CH₃

—NH(CH₂)₈CH═CHCH₂CH═CH(CH₂)₄CH₃

—NH(CH₂)₈(CH═CHCH₂)₃CH₃

—NH(CH₂)₈CH═CH(CH₂CH═CH)₃(CH₂)₄CH₃

—NH(CH₂)₈CH═CH(CH₂)₅CH₃ Trans

—NH(CH₂)₈CH═CH(CH₂)₇CH₃ Trans

Compounds of the present invention may be prepared from readily available starting materials using synthetic peptide chemistry well known to the skilled person. The scheme shown in FIG. 1 shows a general scheme for the synthesis of the compounds of the invention wherein the aminohydrocarbyl groups are linked to the diaminodicarboxylic acid moeity by amide bonds, the scheme shown in FIG. 2 shows a general scheme for the synthesis of the compounds of the invention wherein the head group is a diacid linked to the α-amino group of diaminoacid moeity by amide bonds and the scheme shown in FIG. 3 shows a general scheme for the synthesis of the compounds of the invention wherein the head group is a diacid linked to the non-α-amino group of a diaminoacid moeity by amide bonds.

Another aspect of the invention relates to methods for using diaminodicarboxylic acid:peptide-based gemini compounds. Such uses include facilitating the transfer of oligonucleotides and polynucleotides into cells for antisense, gene therapy and genetic immunisation (for the generation of antibodies) in whole organisms. Other uses include employing the compounds of the invention to facilitate the transfection of polynucleotides into cells in culture when such transfer is required, in, for example, gene expression studies and antisense control experiments among others. The polynucleotides can be mixed with the compounds, added to the cells and incubated to allow polynucleotide uptake. After further incubation the cells can be assayed for the phenotypic trait afforded by the transfected DNA, or the levels of mRNA expressed from said DNA can be determined by Northern blotting or by using PCR-based quantitation methods for example the Taqmar method (Perkin Elmer, Connecticut, USA). Compounds of the invention offer a significant improvement, typically between 3 and 6 fold, in the efficiency of cellular uptake of DNA in cells in culture, compared with compounds in the previous art. In the transfection protocol, the gemini compound may be used in combination with one or more supplements to increase the efficiency of transfection. Such supplements may be selected from, for example:

(i) a neutral carrier, for example dioleyl phosphatidylethanolamine (DOPE) (Farhood, H., et al (1985) Biochim. Biophys. Acta 1235 289);

(ii) a complexing reagent, for example the commercially available PLUS reagent (Life Technologies Inc. Maryland, USA) or peptides, such as polylysine or polyornithine peptides or peptides comprising primarily, but not exclusively, basic amino acids such as lysine, ornithine and/or arginine. The list above is not intended to be exhaustive and other supplements that increase the efficiency of transfection are taken to fall within the scope of the invention.

In still another aspect, the invention relates to the transfer of genetic material in gene therapy using the compounds of the invention.

Yet another aspect of the invention relates to methods to effect the delivery of non-nucleotide based drug compounds into cells in vitro and in vivo using the compounds of the invention.

The following definitions are provided to facilitate understanding of certain terms used frequently herein.

“Amino acid” refers to dipolar ions (zwitterions) of the form ⁺H₃NCH(R)CO₂—. They are differentiated by the nature of the group R, and when R is different from hydrogen can also be asymmetric, forming D and L families. There are 20 naturally occurring amino acids where the R group can be, for example, non-polar (e.g. alanine, leucine, phenylalanine) or polar (e.g. glutamic acid, histidine, arginine and lysine). In the case of un-natural amino acids R can be any other group which is not found in the amino acids found in nature.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Transfection” refers to the introduction of polynucleotides into cells in culture using methods involving the modification of the cell membrane either by chemical or physical means. Such methods are described in, for example, Sambrook et al., MOLECULAR CLONING: A LABORATORY MANUAL, 2nd Ed., Cold Spring Harbor Laboratory Press, Cold Spring Harbor, N.Y. (1989). The polynucleotides may be linear or circular, single-stranded or double-stranded and may include elements controlling replication of the polynucleotide or expression of homologous or heterologous genes which may comprise part of the polynucleotide.

The invention will now be described by way of the following examples.

### EXAMPLES

### Example 1

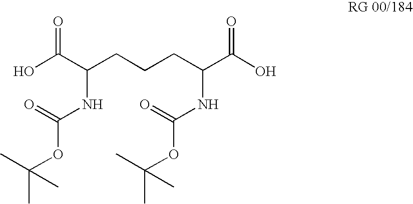

D,L-α,ε-diaminopimelic acid (29.0 mmol; 5.52 g) was dissolved in THF/water 1/1 (50 ml) then NaOH (11.6 mmol; 2.55 g; 2.2 eq.) and Boc₂O (11.6 mmol; 13.94 g; 2.2 eq.) were added. The mixture was stirred overnight at room temperature. Most of the THF was removed and the mixture was acidified to pH 2 with HCl 3 M. The precipitate was extracted twice with chloroform then organic layers were combined and washed successively with water and brine, dried over anhydrous sodium sulfate and evaporated to yield the bis-protected compound (9.52 g, 84%/o).

### Example 2

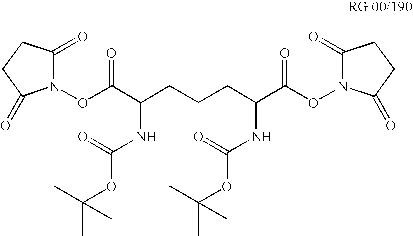

The bis-Bocdiaminopimelic acid (7.07 g, 18.1 mmol) of example 1 was dissolved in THF (160 mL) then N-hydroxysuccinimide (4.34 g, 37.7 mmol, 2.1 eq.) and DCC (7.62 g, 36.9 mmol; 2.04 eq.) were added. The mixture was stirred 20 h at room temperature. The precipitate was filtered off and washed with EtOAc. Solvents were removed and the residue redissolved in EtOAc, cooled to 0° C. and filtered. After evaporation of the solvent, Et₂O is added to precipitate the solid. After filtration of Et₂O, a white solid (9.81 g, 93% Yo) is obtained. Mass spectrum (+ESI): 607.2231 (M+Na).

### Example 3

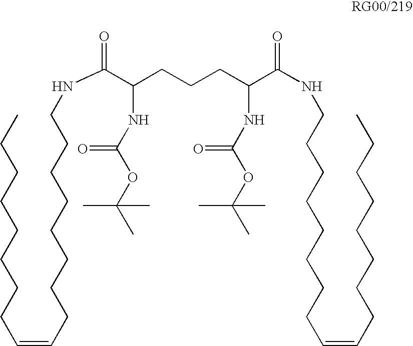

The bis-activated diaminopimelic derivative (1.48 g, 2.53 mmol) of example 2 was dissolved in THF (60 mL) then oleylamine 2.2 eq. (1.49 g, 5.57 mmol) and K₂CO₃ 2.2 eq. (0.77 g, 5.57 mmol) in 5 ml of water were added. The mixture was left stirring at room temperature for 24 h. Most of the THF was removed by evaporation then water (100 mL) was added. The mixture was extracted with CHCl₃ (2×60 ml). The combined organic layers were washed with brine, dried over Na₂SO₄ and evaporated. The residue was purified by chromatography on silica gel with ether/dichloromethane (1/2, v/v, rf-0.3) to yield a white solid foam (1.68 g, 75%). Mass spectrum (+ESI): 911.7554 (M+Na).

### Example 4

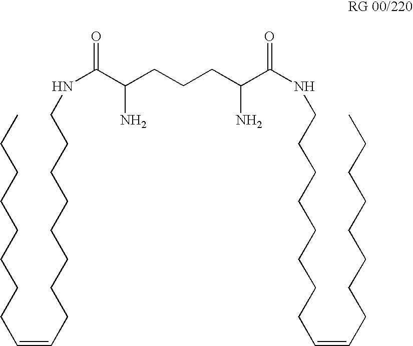

The di-Boc protected RG 00/219 of example 3 (1.50 g, 1.69 mmol) was stirred in a 1/1 mixture of CH₂Cl₂/TFA (20 mL) for 1 h. The solvent were evaporated. The oily residue was diluted in CHCl₃ and washed successively with 1M K₂CO₃, water and brine, dried on sodium sulfate, filtered and evaporated to give a yellowish solid. Trituration in Et₂O followed by filtration gave a white powder (1.15 g, 99%).

### Example 5

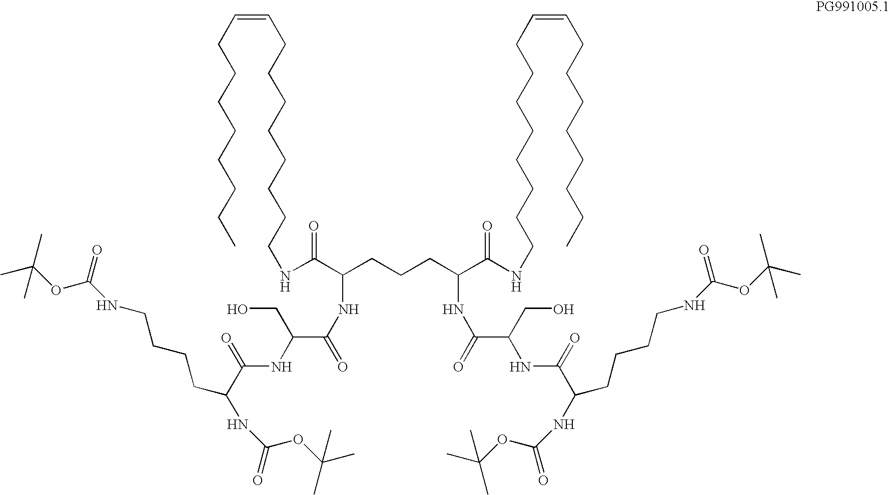

The diamine PG32788 100 mg (0.11 mmol) was dissolved in THF (10 ml) then potassium carbonate 2.2 eq. (34 mg; 0.24 mmol) in water (2 ml) and the Nα,Nε-bis-ter-butyl-carbamate-L-lysine-L-serine-O-succinimidate (was built by the usual peptide synthesis) 2.05 eq. (0.22 mmol; 119 mg) in THF (8 ml) were added. The mixture was stirred overnight at room temperature. Most of the THF was removed then water (15 ml) was added and the precipitate was extracted with chloroform (2×25 ml). Organic phases were combined and washed with 4% NaHCO₃ (15 mL), water (15 mL), 4% citric acid (15 mL), water (15 mL), brine (15 ml), dried over sodium sulfate and concentrated to yield 160 mg (96.%) of coupling compound.

### Example 6

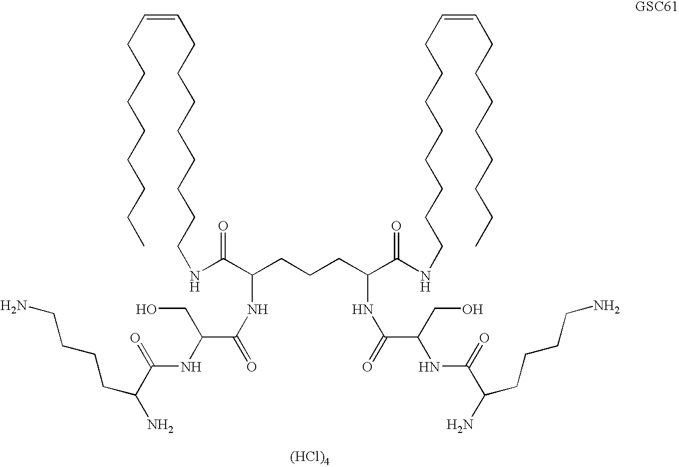

PG991005.1 from example 5 (155 mg, 0.102 mmol) was dissolved in a mixture of methanol/conc. HCl 1/1 (20 ml). The mixture was stirred 2 h at room temperature. Solvent was removed and crude product was redissolved in water (80 ml), filtrated on sintered frit funnel (N° 3) then freeze dry to yield 108 mg (83%) of GSC61.

### Example 7

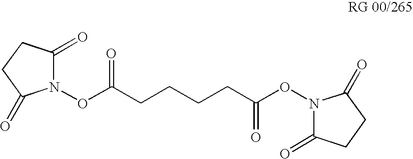

To a solution of adipoyl chloride (3.66 g, 20 mmol) in tetrahydrofuran (180 ml) were added N-hydroxysuccinimide (4.60 g, 40 mmol, 2 eq.) and triethylamine (5.69 ml, 40 mmol, 2 eq.). After 24 h at room temperature, the solvent was evaporated and the residue was partinated between dilute aqueous HCl and chloroform. The organic layer was extracted and washed successively with water and brine, dried over sodium sulphate, filtered and evaporated to give a white solid (5.99 g, 88%).

NMR ¹H (CDCl₃): δ 1.69 (m, 2H, CH₃), 2.69 (m, 2H), 2.78 (s, 4H, succinimide)

### Example 8

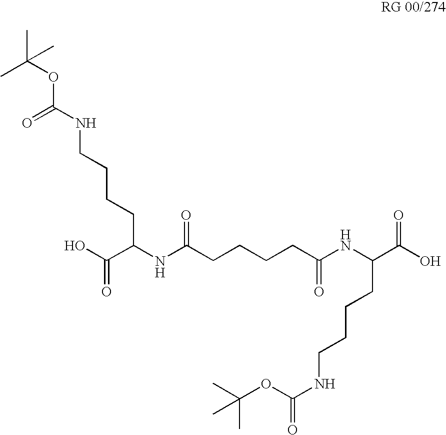

To a stirred solution of adipoyl disuccinimidate (1.50 g, 4.41 mmol) in tetrahydrofuran (150 mL) were added the α- or β-Boc-protected lysine (2.17 g, 8.82 mmol, 2 eq.) and potassium carbonate (1.30 g, 9.40 mmol, 2.1 eq.) in 30 ml of water. The reaction is stirred at room temperature for 20 h. Most of the ThF was removed and the mixture was acidified to pH 2 with 3 M HCl. The precipitate was extracted twice with chloroform then organic layers were combined and washed successively with water and brine, dried over anhydrous sodium sulfate and evaporated to give a white powder (2.66 g, 77%).

### Example 9

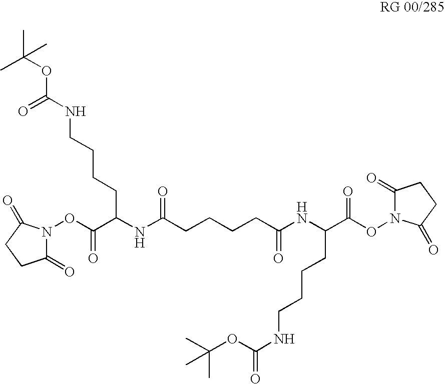

To a solution of di-N-(N-8-tert-butylcarbonate-lysinyl)adipate (2.30 g, 3.82 mmol) in tetrahydrofuran (180 mL) were added N-hydroxysuccinimide (0.90 g, 7.80 mmol, 2.05 eq.) and DCC (1.57 g, 7.66 mmol, 2 eq.). The mixture was stirred for 24 h at room temperature and DCU was filtered and washed with EtOAc (3×30 mL). The solvents were evaporated and the residue dissolved in EtOAc (40 mL) and the precipitate filtered. After evaporation, the oily was cristallised in Et₂O to give a white powder (2.80 g, 92%).

### Example 10

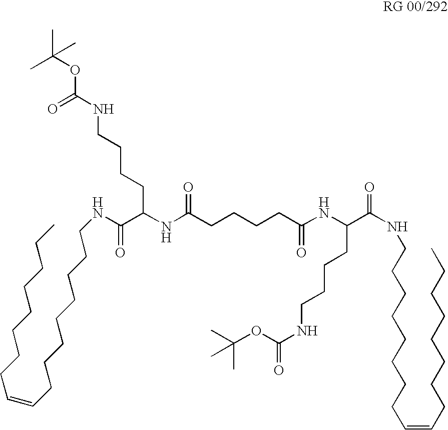

To a solution of the succinimidyl ester RG 00/285 of example 9 (1.50 g, 1.88 mmol) in THF (80 mL) were added oleylamine (1.01 g, 3.78 mmol, 2.02 eq.) and potassium carbonate (0.53 g, 3.83 mmol, 2.1 eq.) in water (10 mL). The reaction was stirred for 20 h at room temperature. Most of the THF was removed and the mixture was partionated between water and chloroform. The organic layer was extracted and washed successively with water, 1M HCl, water and brine. After drying over sodium sulfate, filtration and evaporation, the residue was purified by chromatography on silica gel in CHCl₃/MeOH (97/3, v/v, Rf: 0.33) to give an oil (1.35 g, 65%). Mass spectrum (+ESI): 1123.89793 (M+Na).

### Example 11

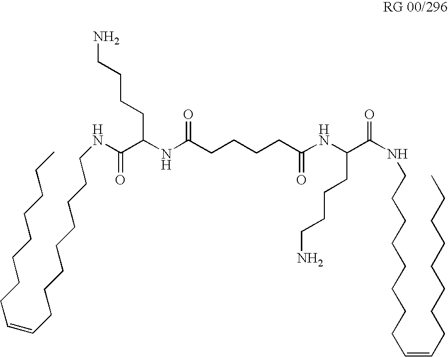

The di-Boc protected RG 00/292 of example 10 (1.20 g, 1.09 mmol) was stirred in a 1/1 mixture of CH₂Cl₂/TFA (20 mL) for 1 h. The solvent were evaporated. The oily residue was diluted in CHCl₃ and washed successively with 1M K₂CO₃, water and brine, dried on sodium sulfate, filtered and evaporated to give a yellowish solid. Trituration in Et₂O followed by filtration gave a white powder (0.95 g, 97%). Mass spectrum (+ESI): 901.82280 (MFI+).

### Example 12

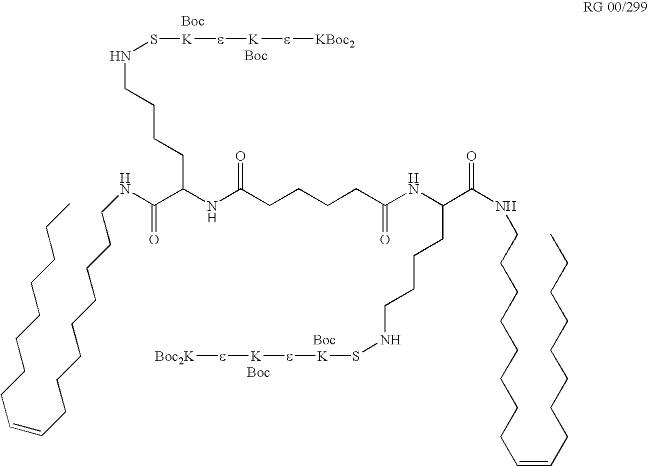

To a solution of the diamino RG 00/296 from example 11 (228 mg, 0.25 mmol) and K₂CO₃ (74 mg, 0.54 mmol, 2.1 eq.) in a 9/1 THF/water mixture (40 mL, v/v) was added the activated peptide Boc₂K-ε-K(Boc)-ε-K(Boc)-S—OSu (500 mg, 0.5 mmol, 2 eq.). The reaction was stirred for 24 h at room temperature. Most of the THF was removed under vaccuum. A 4% Na₂CO₃ solution (10 mL) was added and the aqueous layer was extracted with CHCl₃ (3×40 mL). The combined organic layers were washed successively with water (20 mL), 4% citric acid (20 mL), water (20 mL), brine (20 mL), dried over sodium sulfate, filtered and evaporated to give a pale yellowish solid (543 mg, 81%). Mass spectrum (+ES): 2666.848 (M+Na).

### Example 13

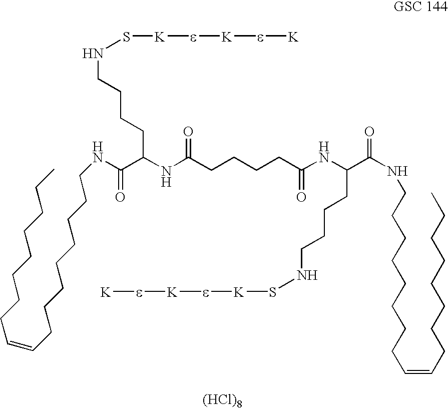

To a solution of Boc-protected gemini RG 00/299 from example 12 (500 mg, 0.19 mmol) in MeOH (10 mL) was added concentrated aqueous HCl (10 mL). The reaction was stirred for 1.5 h at room temperature. The solvents were evaporated and the residue dissolved in water (80 mL), filtered on a N°3 frit sinter. The aqueous layer was evaporated to dryness. The residue was dissolved in MeOH (4 mL) and Et₂O was added. The precipitate was collected to give a pale pink powder (348 mg, 86%). Mass spectrum (+ESI): 1844.446 (MH⁺).

### Example 14

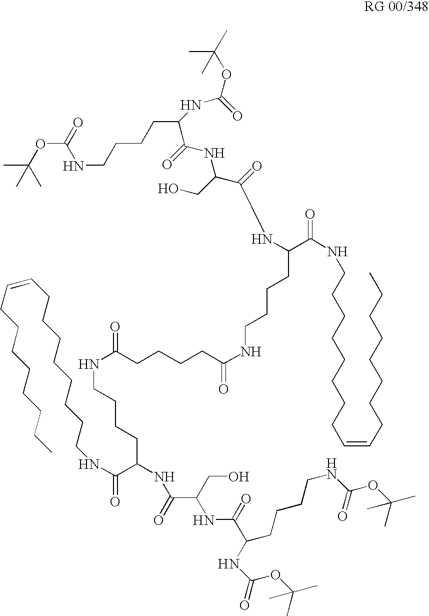

To a solution of diamino derivative from example 13 (255 mg, 0.28 mmol) and potassium carbonate (82 mg, 0.59 mmol, 2.1 eq.) in a 9/1 mixture of THF/water (80 mL) was added Nα,Nε-bis-ter-butyl-carbamate-L-lysine-L-serine-O-succimnimdate (300 mg, 5.65 mmol, 2.05 eq.). The reaction was stirred at room temperature for 20 h. Most of the THF was removed by evaporation and the aqueous residue diluted with 4% NaHCO₃ (20 mL). The aqueous layer was then extracted twice with CHCl₃ (25 mL). The combined organic layers were washed successively with water (110 mL), 0.011 M HCl (110 mL), water (115 mL) and brine (20 mL), dried over sodium sulfate, filtered and evaporated to give a white solid (462 mg, 94%).

### Example 15

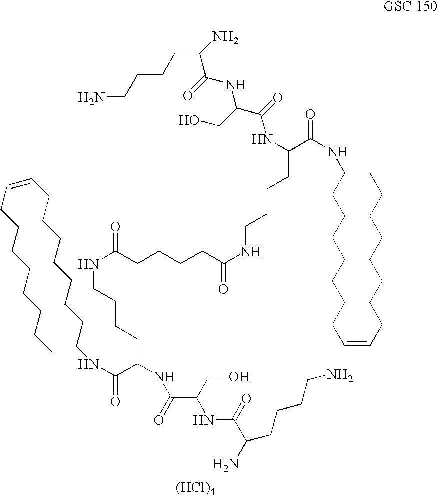

To a solution of protected gemini RG 00/348 from example 14 (441 mg, 0.25 mmol) in MeOH (10 mL) was added concentrated HCl (10 mL). The mixture is then stirred for 1 h at room temperature. The solvents were evaporated to dryness. The residue was redissolved in water (80 mL) and filtered on a sintered frit funnel (N° 3) and evaporated using ethanol as co-solvant. The residue was then dissolved in MeOH (5 mL) and Et₂O was added until complete precipitation. The solid was isolated by decantation and dried under high vaccuum to give a yellowish powder (341 mg, 91%).

### Example 16

### Compound GSC 112

Compound GSC 112 is an aminopimelic gemini compound, synthesised according to the schemes described herein and has the structure:

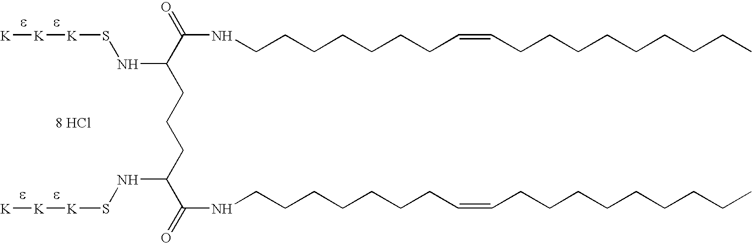

### Example 17

### Transfection of Recombinant Plasmid Expressing Luciferase Into Cells Using Aminopimelic and Adipate-Lysine:Peptide-based gemini surfactant Compounds

Transfection studies were performed using the adherent cell line CHO-K1 (Puck et al. 1958). Complete medium consisted of MEM alpha medium supplemented with 10% v/v foetal bovine serum and 1×L-Glutamine. All media and supplements were obtained from Life Technologies.

In Vitro Gene Transfection.

Cells were seeded into 96-well MTP plates (Nunc) 16-18 hours prior to transfection at an approximate density of 1×10⁴ cells per well. For transfection, 0.064 μg of the luciferase reporter gene plasmid, pGL3-Control Vector (Promega) per well, was incubated with various concentrations of the gemini compounds GSC112 or GSC150 and complexing agents in a final volume of 100 μl. After 30 minutes incubation at RT, OPTI-MEM® medium (Life Technologies) was added to the transfection mixture and the solution placed on the cells (final volume per well: 100 μl). Following a 3 hour or over night incubation at 37° C., the transfection solution was replaced with complete medium and the cells incubated further at 37° C. Reporter gene assays were performed according to the manufacturer's guidelines (Roche Diagnostics) approximately 48 hours post transfection. Luminescence was measured in a Packard TopCount NXT Microplate Scintillation and Luminescence Counter.

### BRIEF DESCRIPTION OF THE DRAWINGS

FIG. 1. General sheme for synthesis of compounds of the invention wherein the aminohydrocarbyl groups are linked to the diaminodicarboxylic acid acid moeity by amide bonds.

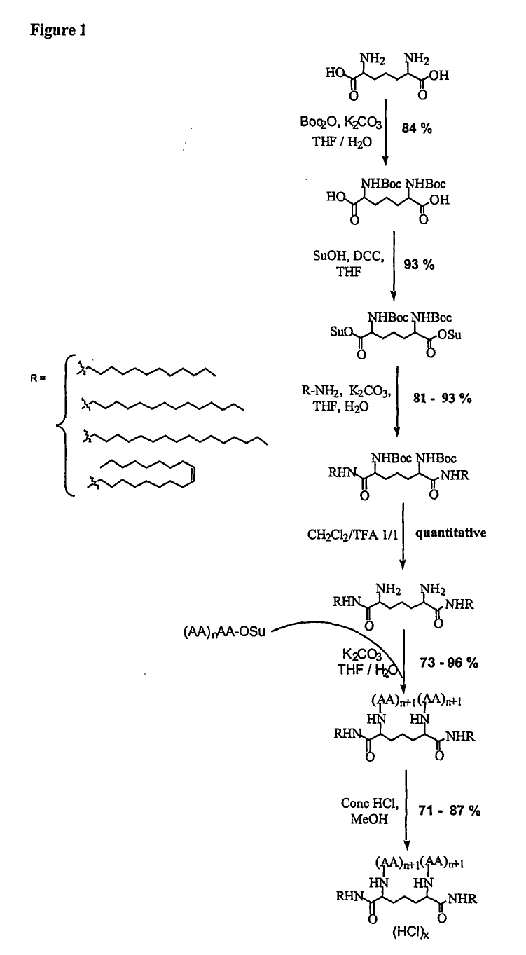

FIG. 2. General sheme for synthesis of compounds of the invention wherein the head group is a diacid linked to the α-amino group of the diaminoacid moeity by amide bonds.

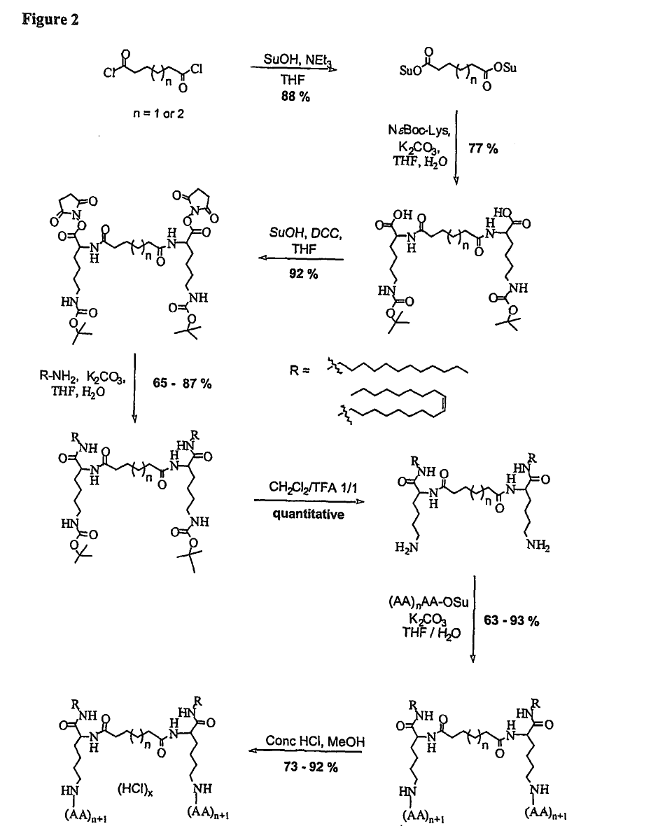

FIG. 3. General sheme for synthesis of compounds of the invention wherein the head group is a diacid linked to the non-α-amino group of a diaminoacid moeity by amide bonds.

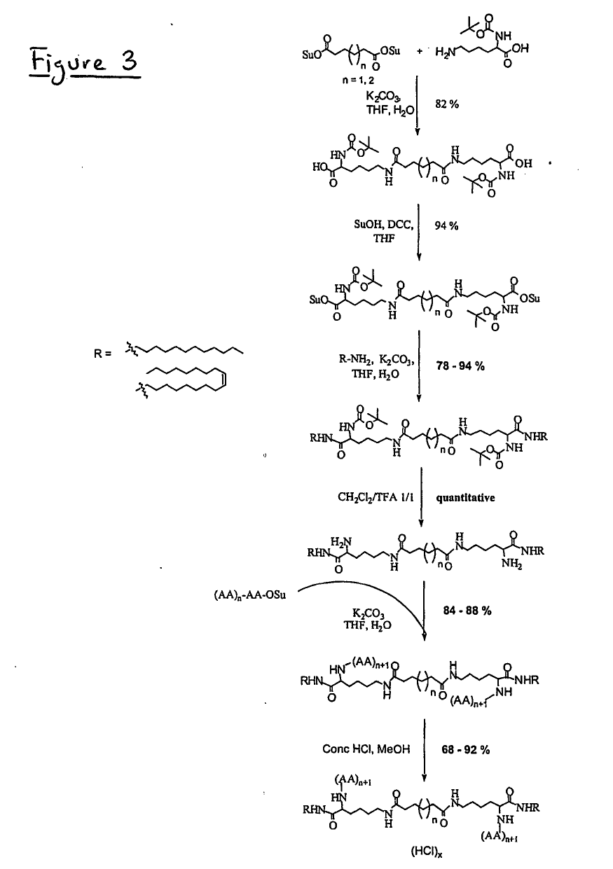

FIG. 4. Transfection of CHO-KI cells with gemini surfactant GSC 112. The numbers along the x axis refer to concentration of gemini compounds in μM. The block of 5 bars at the right of the chart show the data obtained when the DNA was premixed with polylysine. The block of 5 bars at the left side of the chart show data when no polylysine was used. The figures on the Y axis represent CPS (count per second) from the luciferase assay. The bars represent the mean CPS (count per second) of 4 experiments ±the standard error of the mean.

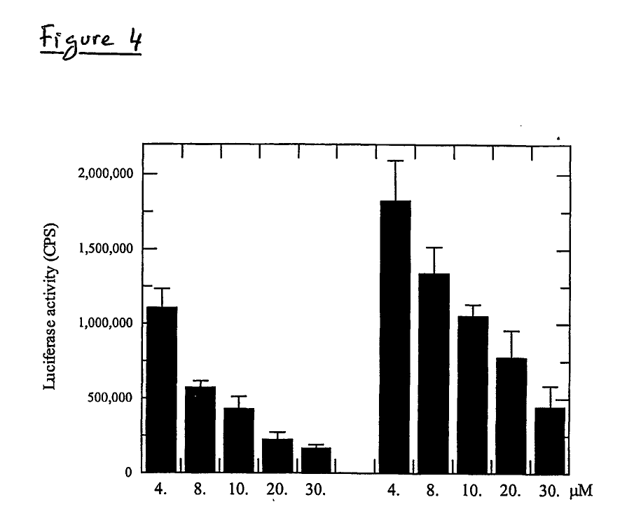

FIG. 5. Transfection of CHO-K1 cells with gemini surfactant GSC150. The numbers along the x axis refer to concentration of gemini compounds in μM. The block of 5 bars at the right of the chart show the data obtained when the DNA was premixed with polylysine. The block of 5 bars at the left side of the chart show data when no polylysine was used. The figures on the Y axis represent CPS (count per second) from the luciferase assay. The bars represent the mean CPS (count per second) of 4 experiments ±the standard error of the mean.

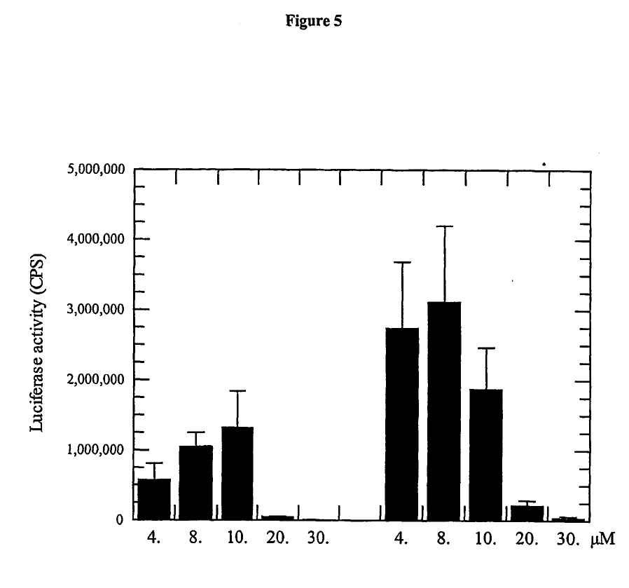

1. A diaminodicarboxylic acid:peptide-based gemini compound having a diaminodicarboxylic acid backbone and conforming to the general structure of formula (I): 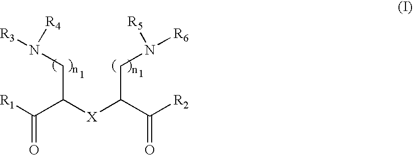
where X=(CH₂)ₙ₂, n2 is 1 to 8 and n1 is 0; or
where X=NHC(O)(CH₂)ₙ₃C(O)NH, n3 is 1 to 8 and n1 is 2 to 4; or
where X=(CH₂)ₙ₄NHC(O)(CH₂)ₙ₅C(O)NH(CH₂)ₙ₄, n4 is 2 to 4, n5 is 1 to 8 and n1 is 0; and
where R3, R4, R5 and R6 is hydrogen; or
where R₃ and R₅ is hydrogen and R₄ and R₆ which may be the same or different are peptide groups formed from one or more amino acids linked together by amide (CONH) bonds and further linked to the diaminodicarboxylic acid backbone by amide bonds, in a linear or branched manner, having the general formula (ii): 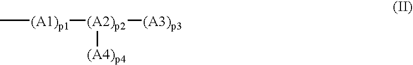
where the values for p1 and p2, which may be the same or different, are from 0 to 5, preferably 1; and
the values for p3 and p4, which may be the same or different, are from 0 to 5, preferably 0;
A1, A3 and A4, which may be the same or different, is an amino acid selected from serine, lysine, omithine, threonine, histidine, cysteine, arginine and tyrosine; and
A2 is an amino acid selected from lysine, omithine and histidine; or
where R₄ and R₆, which may be the same or different, is a group having the formula (III): 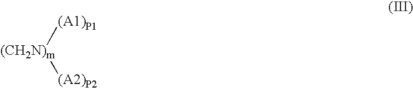
and R₃ and R₅, which may be the same or different, is a group having the formula (IV): 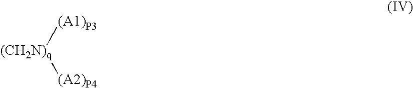
where p1, p2, p3, and p4 which may be the same or different, are 0 to 5;
and where m is 0 to 5; q is 1 to 5;
and where A1 to A4 are as defined above;
and R₁ and R₂ are saturated or unsaturated aminohydrocarbyl groups having up to 32 carbon atoms and linked to the diaminodicarboxylic acid backbone by an amide bond; or
a salt, preferably a pharmaceutically acceptable salt thereof.

2. A diaminodicarboxylic acid:peptide-based gemini compound according to claim 1 which is symmetrical, that is R₁ and R₆ are the same, R₂ and R₄ are the same, and R₃ and R₅ are the same.

3. A diaminodicarboxylic acid:peptide-based gemini compound according to claim 1 or 2 wherein in the peptide group of formula (1H) p1 and p2 are both 1 and p3 and p4 are both 0.

4. A diaminodicarboxylic acid:peptide-based gemini compound according to any one of claims 1 to 3 wherein the A1 is serine.

5. A diaminodicarboxylic acid:peptide-based gemini compound according to any one of claims 1 to 4 wherein the A2 is lysine.

6. A diaminodicarboxylic acid:peptide-based gemini compound according to claim 1 wherein the aminohydrocarbyl group is selected from:
—NH(CH₂)₁₁CH_{3 —NH(CH}₂)₁₃CH₃
—NH(CH₂)₁₅CH₃
—NH(CH₂)₁₇CH₃
—NH(CH₂)₁₉CH₃
—NH(CH₂)₂₃CH₃
—NH(CH₂)₈CH═CH(CH₂)₅CH₃
—NH(CH₂)₈CH═CH(CH₂)₇CH₃
—NH(CH₂)₈CH═CHCH₂CH═CH(CH₂)₄CH₃
—NH(CH₂)₈(CH═CHCH₂)₃CH₃
—NH(CH₂)₄CH═CH(CH₂CH═CH)₃(CH₂)₄CH₃
—NH(CH₂)₈CH═CH(CH₂)₅CH₃ Trans
—NH(CH₂)₈CH═CH(CH₂)₇CH₃ Trans
—NH(CH₂)₉CHCH₃(CH₂)₇CH₃

7. A diaminodicarboxylic acid:peptide-based gemini compound according to claim 1 wherein the aminohydrocarbyl group is selected from:
—NH(CH₂)₁₁CH₃
—NH(CH₂)₁₃CH₃
—NH(CH₂)₁₅CH₃
—NH(CH₂)₁₇CH₃
—NH(CH₂)₁₉CH₃
—NH(CH₂)₂₃CH₃
—NH(CH₂)₈CH═CH(CH₂)₅CH₃
—NH(CH₂)₈CH═CH(CH₂)₇CH₃
—NH(CH₂)₈CH═CHCH₂CH═CH(CH₂)₄CH₃
—NH(CH₂)₈(CH═CHCH₂)₃CH₃
—NH(CH₂)₄CH═CH(CH₂CH═CH)₃(CH₂)₄CH₃
—NH(CH₂)₈CH═CH(CH₂)₅CH₃ Trans
—NH(CH₂)₈CH═CH(CH₂)₇CH₃ Trans
—NH(CH₂)₉CHCH₃(CH₂)₇CH₃
—NHCH₁₇CHOH(CH₂)₂CH₃
—N((CH₂)₁₅CH₃)₂

8. The compound:

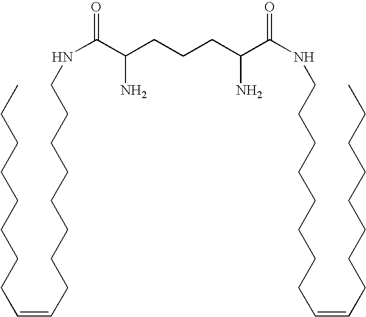

9. The compound:

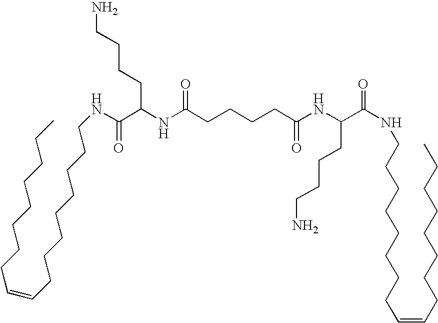

10. The compound:

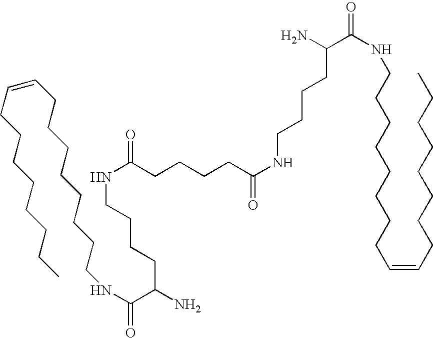

11. The compound GSC61 of formula:

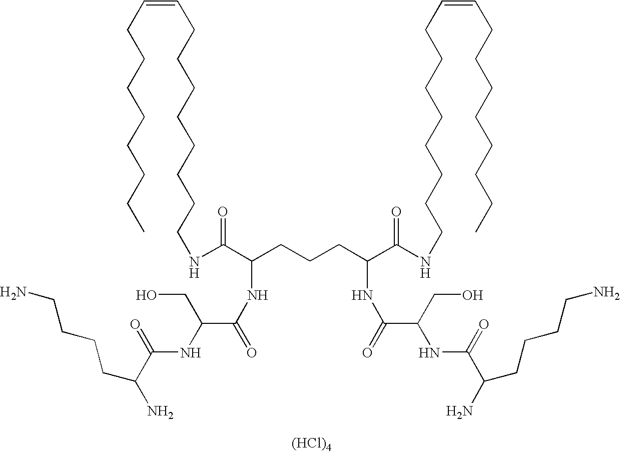

12. The compound GSC 144 of formula:

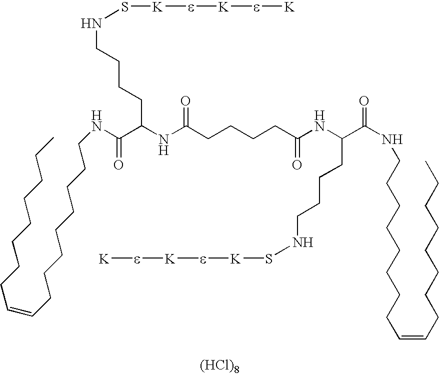

13. The compound GSC150 of formula:

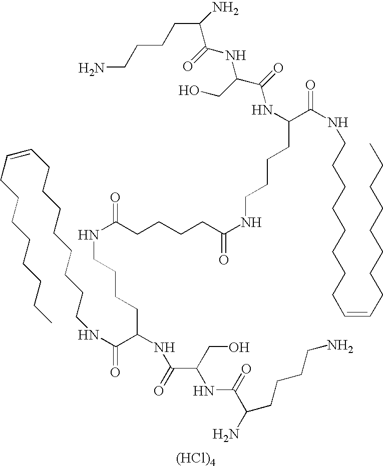

14. The use of a diaminodicarboxylic acid:peptide-based gemini compound according to claim 1 to effect the delivery of polynucleotides into cells in vitro and in vivo.

15. The use of a diaminodicarboxylic acid:peptide-based gemini compound according to claim 1 to effect the delivery of non-nucleotide based drug compounds into cells in vitro and in vivo.

16. The use of a diaminodicarboxylic acid:peptide-based gemini compound according to claim 14 or 15 wherein the compound is used in combination with one or more supplements selected from the group consisting of:
(i) a neutral carrier; or
(ii) a complexing reagent.

17. The use according to claim 16 wherein the neutral carrier is dioleyl phosphatidylethanolamine (DOPE).

18. The use according to claim 17 wherein the complexing reagent is selected from the group consisting of:
i) PLUS reagent;
ii) a peptide comprising mainly basic amino acids;
iii) a peptide consisting of basic amino acids;
iv) a peptide consisting of basic amino acids selected from lysine and arginine.
